(19) United States
(12) Patent Application Publication
(10) Pub. No.: US 2022/0087515 A1
(43) Pub. Date: Mar. 24, 2022

(54) ENDOSCOPE SYSTEM

(71) Applicant: Qioptiq Photonics GmbH & Co. KG, Göttingen (DE)

(72) Inventors: Axel Kasper, Muenchen (DE); Bernhard Lorenz, Marzling (DE)

(21) Appl. No.: 17/425,512

(22) PCT Filed: Feb. 5, 2020
(86) PCT No.: PCT/US20/16702

§ 371 Date: Jul. 23, 2021

Related U.S. Application Data
(60) Provisional application No. 62/801,711, filed on Feb. 6, 2019.

(51) Int. Cl. A61B 1/00; A61B 5/00; A61B 1/05; A61B 1/06

(52) U.S. Cl. CPC A61B 1/00172; A61B 5/0066; A61B 1/05; A61B 1/00114; A61B 1/0638; A61B 1/00096; A61B 1/00193; A61B 1/00149; A61B 1/063; A61B 1/00124

(57) ABSTRACT

An endoscope system (200, 300) for imaging an interior of a patient (180) comprises an endoscope tube (210, 310), an imaging unit (350) for imaging the interior of the patient, wherein the imaging unit is at least partially located inside the endoscope tube, and an optical coherence tomography unit (360), wherein said imaging unit (350) is distinct from the OCT unit (360), and wherein a sample arm (360c) of the OCT unit is at least partially located inside the endoscope tube.

### CROSS-REFERENCE TO RELATED APPLICATION(S)

This application claims the benefit of priority to U.S. provisional patent application No. 62/801,711, entitled ENDOSCOPE SYSTEM and filed on Feb. 6, 2019. The disclosure of the prior application is incorporated herein by reference in its entirety.

### FIELD OF THE INVENTION

The invention relates to an endoscope system.

### BACKGROUND OF THE INVENTION

There are some imaging modalities in medicine such as ultrasonic allowing for imaging an interior of a patient and into depth of a tissue. However, there is a growing knowledge about many diseases down to smallest scales and an additional demand that any examination of a patient should be as least injurious as possible.

Therefore, it is a desire to provide examination methods being non-destructive and having high resolution and liability.

### SUMMARY OF THE INVENTION

According to an embodiment of the invention an endoscope system for imaging a sample, an inner part of a patient, or an organ, comprises: an endoscope tube, an imaging unit for imaging the inner part of the patient, wherein the imaging unit is at least partially located inside the endoscope tube, and an optical coherence tomography (OCT) unit, wherein said imaging unit is distinct from the OCT unit, and wherein a sample arm of the OCT unit is at least partially located inside the endoscope tube.

According to an embodiment of the invention a method of operating an endoscope system for imaging a sample, an inner part of a patient, or an organ, comprises:
providing an endoscope tube,
providing an imaging unit for imaging the inner part of the patient, wherein the imaging unit is at least partially located inside the endoscope tube, and
providing an optical coherence tomography unit, wherein said imaging unit is distinct from the OCT unit, wherein a sample arm of the OCT unit is at least partially located inside the endoscope tube, and
processing data from the imaging unit and/or from the OCT unit, so that the processed data are displayable on a screen.

The expression “endoscope system” may refer to a system for imaging the inner part of the patient including an endoscope tube. Said endoscope system may comprise at least two imaging modalities.

The expression “endoscope tube” may refer to a tubular patient interface being inserted into the patient. The endoscope tube may be rigid or flexible. The endoscope tube may have a typical diameter in the range between 5 mm and 12 mm, or even less. At its distal end, the endoscope tube may comprise a tube head being rigid.

The expression “imaging unit for imaging the inner part of the patient” may refer to an imaging modality, or imaging channel which could be of the type of a combination of: 2D or 3D, extended depth of field imaging (EDOF), light field imaging, pupil plane encoding, Camera CCD, CMOS, Laser scanning, VIS, NIR, fluorescence imaging, hyperspectral imaging, or any combination thereof. The imaging unit may comprise an image acquisition device and a lens, which both may be arranged within the endoscope tube, or within the tube head. A presentation of the imaging unit may be a camera image, being a two-dimensional presentation of data displayed as a top or planar view of the sample, e.g. on a live screen.

The expression “OCT unit for imaging the inner part of the patient” may refer to an imaging modality comprising a light source arm, a reference mirror arm, a sample arm, and a detector arm. The reference arm may be configured such that the reference arm has the same optical path length and include optic media that provide the same spectral dispersion. A displayable result, such as a B-scan or C-scan, may be shown on a screen. The B- or C-scans may be based on interference data being measured on a detector at the end of the detector arm and by scanning the sample (inner part of the patient) along a line (B-Scan) or in a certain area (C-scan). The OCT unit is different from the imaging unit. The OCT unit may be a combination of at least one of the group of using a light source such as so called NIR (750-1400 nm and above wavelength), or VIS (400-750 nm wavelength), SLED light source (super-luminescent diode), FDML laser (frequency-domain mode-locked laser), or super-continuum light source. Further, in addition, the OCT unit may comprise a scanning element like a MEMS scanner for 1D or 2D scanning (such as: Resonant, Closed-loop, Combination of both), galvanometric scanners, an oscillating fiber scanner, or a rotating prism scanner. Furthermore, the OCT unit may comprise a detector (for a so-called A-scan) of at least one of the group of spectrally resolved spectrometer (with line sensor), or a time resolved (swept source) spectral detection with a photo detector like e.g. an avalanche photo diode (APD), or a silicon photo-multiplier (SiPM). An OCT unit can image a depth of several millimeters at a time depending on the optical properties of the tissue. All aspects of the present application could be applied to any type of OCT system, including, but not limited to time-domain (TD-OCT), spectral-domain (SD-OCT), and swept-source (SS-OCT).

The expression “inner part of the patient” expresses that a distal end of the endoscope tube may be located within the patient or a part of the patient, and may also express that an endoscope head may be close to tissue of the patient even if the endoscope head or the distal end of the endoscope is not completely enclosed by the patient's tissue. This may, e.g., apply to brain surgery, when the endoscope head approaches brain tissue in a hollow space of the patient's head.

A functioning of the OCT unit may be based on a building structure similar to a, e.g. Michelson-Interferometer, an Interferometer having 4 functional arms (a light source arm, a reference mirror arm, a sample arm, and a detector arm), so that light coming from a reference arm and light from a sample arm may interfere with each other. The sample and reference arms in the interferometer could consist of free-space optics, bulk-optics, fiber-optics or combinations thereof and could have different interferometer architectures not only such as Michelson, but also Mach-Zehnder, or common-path based designs as would be known by those skilled in the art. Light beam, or OCT beam, as used herein should be interpreted as any carefully directed light path.

The expression “wherein a sample arm of the OCT unit is at least partially located inside the endoscope tube” refers to a part of the OCT beam path being directed to the sample. The OCT beam being directed towards the sample or in other words “the sample arm” of the OCT is therefore partially protected by the endoscope tube while imaging is generated. The endoscope tube may usually have a typical diameter in the range between 5 mm and 12 mm, or even less.

The endoscope system may be an en-face endoscope generating pictures from an opposite side of the patient, or viewing point inside the patient. The viewing point may allow for directly focusing on a surface of a sample resulting in a field of view. By scanning the examined surface in order to achieve more data about a broader area the imaging unit may serve as a reference by generating an en-face image of the surface of the sample. These reference frontal sections of the sample may be displayed on a screen together with a B-Scan and/or C-Scan provided by the OCT unit covering the same or at least a similar field of view. This may give an operating doctor more detailed information about an interesting area or section of the body.

The used light source of an OCT unit may have an image depth of three or four millimeters into the tissue, or even more. A procedure of scanning as a function of depth is called an axial scan, or “A-scan” which is directed into the depth of a certain tissue area. A data set of A-scans measured at neighboring locations in the sample produces a cross-sectional image (slice, tomogram, or B-scan) of the sample for the respective location. An OCT cross-sectional image provides an image of the tissue which is comparable with a histology of the tissue. Typically, a B-scan may be collected along a straight line or in a generally flat surface, but B-scans generated from scans of other geometries including circular and spiral patterns are also possible. A C-scan may be built up from a large number of A-scans taken from a specific area (en face area) that means that a C-scan comprises information from an examined volume part. Thus, a C-scan may be built up from a set (or row) of different B-scans. It is a commonly accepted convention to define the areal “en-face” view with an area extending in the x- and y-axis. The direction of the A-scan into depth may be defined with the direction of a z-axis. Hence, a picture of a B-scan represents information from a cross-section extending along the z-axis and at least one (or both) of the x- and y-axis. As a consequence, a C-scan represents information from a volume, therefore extending in all different axes (x-, y- and z-axis). Representations of the scans may include, e.g. contour lines, holographic representations and the like.

According to an exemplary embodiment the endoscope system further comprises a screen for displaying one or more images based on processed data from the OCT unit and/or data from the imaging unit.

According to an exemplary embodiment the endoscope system comprises an OCT unit cable and/or an imaging unit cable. The OCT unit cable may comprise a fiber optic cable as a part of the sample arm of the OCT unit, and the imaging unit cable may couple the endoscope tube with an image processing unit.

The OCT unit cable may include the control data cable (controlling the micro scanner) and the fiber optic cable, which may be a single mode fiber.

According to an exemplary embodiment the endoscope system comprises a connection cable, wherein the connection cable comprises the OCT unit cable and the imaging unit cable.

A connection cable may be a flexible cable in which the sample arm of the OCT unit (OCT sample arm) may be included. The OCT sample arm may comprise a fiber optic cable. Further, the connection cable may include a control data cable for controlling a micro scanner being arranged so to scan the sample in a specific area, i.e. that the micro scanner directs the OCT beam so that the sample is scanned in the interesting area. The micro scanner may be operated in that, e.g. in a first direction the movement is resonant or harmonic and in a second direction in that the scanner is directed stepwise. The connection cable may submit the required data for operating the micro scanner. Furthermore, the connection cable may include a data cable connecting to the imaging unit in order to submit imaging data (e.g. from a CCD camera) to the imaging unit.

The OCT unit cable, like the imaging unit cable, may be integrative of the connection cable, or may both run separately. The imaging unit cable may provide a data transmission from an imaging capture device, like a CCD camera, to the imaging unit, where the imaging data may be processed.

A light guide for transmitting light for illumination of the sample may transmit light from a light generator (not shown) towards or into the endoscope handle. The light guide may be a separate cable, or being integrated in the connection cable, in the imaging cable, or in the OCT cable. From the endoscope handle, the light guide may further direct the light to the distal end of the endoscope tube, or as an alternative the light may be coupled into the OCT sample arm to illuminate the sample. The light generator may be arranged at a proximal end outside of the handle and the endoscope tube as a standalone device for generating illumination light.

The connection cable, the OCT unit cable, and the imaging unit cable, including all sub-cables may branch off or be brought together by various connectors at arbitrary regions between or within the endoscope tube, the OCT unit, the imaging unit, or the image processing unit. The OCT unit and the imaging unit may be integrated in a central unit which may couple to a computer displaying results of the C-Scans and B-Scans on a screen.

According to an exemplary embodiment the endoscope system further comprises at least one connector mounted at an end of at least one of the group of the endoscope tube, the connection cable, the OCT unit cable, and the imaging unit cable, so that the endoscope tube is separable from at least one of the group of the OCT unit and the imaging unit.

The endoscope tube may be an exchangeable part within the endoscopes system. In particular, the endoscope tube, respectively the distal end of the optic may be damaged during use and maintenance. Thus, depending on whether the OCT unit cable, the imaging unit cable, and sub-cables may branch off or are grouped together several connectors may allow for disconnecting the endoscope tube and replacing a current one by a new, or refurbished (i.e. maintained and/or cleaned) endoscope tube.

According to an exemplary embodiment of the endoscope system the connector may be pluggable in different orientations, at least in two orientations of 0° and 180°. As a consequence, exchanging of the endoscope tube may be even easier and tolerant against careless use.

According to an exemplary embodiment of the endoscope system the connector may have an inner freedom of rotation, so that the endoscope tube may be rotatable without any restrictions in the degree of rotation.

In particular, this twistable connector may comprise one centered optical path. In particular, the centered optical path may comprise the OCT sample arm. In particular, the optical path of the OCT sample arm may comprise a semitransparent mirror being arranged and comprising a selective transparence so that information or radiation of the OCT sample arm passes freely, and light for illumination and generated by a light source is coupled into the same optical path as of the OCT sample arm. In particular the OCT sample arm and the illumination light use the same optical path from the handle towards the distal end of the endoscope tube, or endoscope head, respectively. The light source may be a halogen light source, a Xenon light source, an LED light source, a laser light source, or the like, and the light may illuminate the sample for imaging, in particular, for stereo imaging. As a practical alternative, the light source may be a distal end of a light guide being fed by the light generator. The light generator may be arranged outside of the handle and the light originating from the light generator may be submitted by the light guide and with the twistable connector. An end of the OCT sample arm and the illumination having the same optical path may comprise a lens being adapted to serve for the OCT sample arm and for illumination at different wavelengths of electromagnetic radiation. The lens of the OCT sample arm being the same of the illumination may be located in direct proximity to one or two lenses for mono or stereo imaging, respectively.

According to a further exemplary embodiment of the endoscope system an offset of a lens of the OCT sample arm in relation to a middle axis of the endoscope tube, or of the endoscope head, respectively, may be compensated by an optical offset compensation. The offset compensation may comprise two prisms or any other kind of optical offset compensation. In particular, the offset compensation works with wavelengths of the electromagnetic waves being used for illumination and being used for the OCT sample arm. By the offset compensation an optical axis representing a center of the optical path of the lens of the OCT sample arm becomes identical with the middle axis of the endoscope tube, or of the endoscope head, respectively, so that the optical axis of the OCT arm becomes identical with the middle axis of the endoscope tube, where the optical axis of the OCT sample arm meets the twistable connector. By this the twistable connector allows for rotating the endoscope tube around its middle axis and avoiding a detrimental deviation of the outer surface of the rotating circular endoscope tube.

In general, a mere rotation of the endoscope tube with the twistable connector may leave the relative positions of the field of view of the OCT system, the field of view of the imaging system, and the illuminated field of view unaltered. This may also apply, when the OCT lens is offset with regards to the middle axis of the endoscope tube, so that the OCT lens may be used for the OCT unit and the illumination when the endoscope tube is rotated with the twistable connector.

According to an exemplary embodiment of the endoscope system the endoscope tube comprises a rigid section, and a micro scanner being located in said rigid section, and/or mounted to said rigid section, wherein the micro scanner is adapted to scan the sample in one and/or two dimensions.

The rigid section of the endoscope tube may also be called “tube head”. The micro scanner may have an option of a rotation built in. That means, if necessary, the micro scanner may be rotated by 90° so that a visual field for the OCT may be enlarged. However, the micro scanner may be located in a section of the endoscope system where the endoscope tube is rigid. It may be helpful that the micro scanner is located in such a rigid section of the endoscope tube, as these strict conditions may be important to bring the OCT beam into interference with the reference arm.

Also, the micro scanner may mutually rotate with the endoscope tube. The rigid section in which the micro scanner is located may also be called endoscope head. A field of view of the OCT (OCT vision field) may itself be off-centric. This may allow for a surgeon to access directly to an interesting region and working directly with surgery tools easier. If the OCT field of view has a smaller diameter than the field of view of the vision unit the rigid end section, or endoscope head, may be moved by rotating the endoscope tube, or the rigid endoscope head may be bent relative to the endoscope tube.

According to an exemplary embodiment of the endoscope system the endoscope tube comprises a flexible section, wherein the flexible section is located behind a rigid tube head seen from a distal end of the endoscope tube, and wherein the micro scanner is located in the rigid tube head or attached to the rigid tube head.

As a consequence, if the micro scanner is located in a rigid section of the OCT sample arm, it is appropriate to mount the micro scanner near to the distal end of the endoscope tube, so that behind the micro scanner (seen from the sample or the distal objective) the OCT sample arm may comprise a fiber optic cable making it possible to let the endoscope tube be flexible towards the OCT unit or imaging unit, respectively. The rigid section may comprise rigid relay optics such as a so-called GRIN (gradient index lens) optics, and/or rod lenses (polished lenses). The flexible section of the endoscope tube may also enable to direct the visual field of the OCT (OCT visual field) towards an interesting area, or volume, respectively.

According to an exemplary embodiment of the endoscope system the endoscope tube comprises an illumination source at its distal end wherein the illumination source is adapted to illuminate the sample with visible light.

An illumination source providing light for capture images being transmitted to the imaging unit may be located at the distal end of the endoscope tube and the illumination source may direct the light to the sample. The term “illumination source” may apply to any sort of providing appropriate light onto the sample in order to capture or generate images in a visible range. In particular, the term illumination may refer to an illumination system applying the principle of the so called “Kohler illumination”.

According to an exemplary embodiment of the endoscope system the endoscope tube comprises a capturing lens and a beam splitter, for providing a shared use of an objective for the OCT unit and the imaging unit, so that an OCT image may be generated with the OCT unit, and a 2D image may be generated with the imaging unit.

If the requirement is to minimize the diameter of the endoscope tube and no 3D imaging is necessary it may be appropriate to use one lens for capturing the reflected beams from the sample, wherein one objective is used, and the reflected beams are split by a beam splitter for providing the OCT unit and the imaging unit in that an OCT image and a 2D image may be generated. The objective may be in shared use for the OCT unit and the imaging unit.

The expression “beam splitter” may refer to a unit, such as a dichroic mirror, or a beam splitter cube, having significantly different reflection or transmission properties at two different wavelengths. The dichroic mirror may be adapted to separate the OCT beam for scanning the sample and being reflected by the sample into an objective at the distal end of the endoscope tube. This may allow for using the identical outmost capturing lens at the distal end of the endoscope tube for both the OCT beam and the beam going to the imaging unit. If the identical outmost capturing lens is used for both units this may be a concept to limit or reduce a diameter of the endoscope tube for the benefit of the patient and his health.

The objective in shared use between the camera imaging and the OCT imaging may be optimized with color-correction for the OCT spectral band and the camera imaging spectral band. If the color correction provides a joint focal plane for both the camera image and the OCT image, then the camera image may be used to find an optimal focus for both imaging modalities at the same time. The optics may also be designed to provide a fixed offset between both focal planes, e.g. to have the optimized camera focus on the tissue surface and the optimized focus for OCT in a certain depth in the tissue (e.g. 1 mm below the tissue surface). Further, the objective in shared use may be equipped with an electronically controlled focusing element, e.g. a liquid lens that allows changing the focal plane/the working distance for both modalities in a synchronized way at the same time.

According to an exemplary embodiment of the endoscope system the OCT image being a 3D representation of a surface area and the image being generated with the imaging unit are processed to represent a 3D image of the surface area. This further processing may apply to the use of one of the lenses being adapted to provide a shared use for the OCT unit and for the imaging unit.

The OCT image being generated with the OCT unit may be a representation of the surface area or of a near surface area. The image being generated with the imaging unit may be a 2D image, or 2D optical image as being visible by usually visible light. In combination, the OCT 3D image and the 2D optical image may be processed to represent a 3D optical image of the (specific) surface area. The surface area of the OCT image and the surface area captured with the imaging unit may partially, widely or completely overlap, so that processing the given information by the 3D OCT image and the 2D image of the imaging unit may provide a sharper 3D surface image compared to generating an image only generated by the OCT unit.

According to an exemplary embodiment of the endoscope system, the endoscope tube comprises a first capturing lens, and a second capturing lens, and a beam splitter, for providing a shared use for one the one of the first and/or second capturing lenses for the OCT unit and/or the imaging unit, so that an OCT image may be generated with the OCT unit, and a 3D image may be generated with the imaging unit.

According to an exemplary embodiment of the endoscope system the OCT image being a 3D representation of a surface area and the image being generated with the imaging unit representing a 3D imaging are processed to represent a 3D image of the surface area. This further processing may apply to pairs of lenses even if the use of one of the lenses provides a shared use for the OCT unit and for the imaging unit. If a 3D imaging is required this could be achieved by a two-lens objective where the first lens may be shared between the OCT unit and the imaging unit by using a beam splitter. A further, second lens may provide a further, second image information for the imaging unit. So, the first lens, and the second lens provide the information needed to create 3D imaging. The first lens fulfills the further task to provide the OCT beam with reflected beams.

The OCT image being generated with the OCT unit may be a representation of the surface area or of a near surface area. The image being generated with the imaging unit may be a 3D image, or 3D optical image as being visible by usually visible light. In combination, the OCT 3D image and the 3D optical image may be processed to represent a 3D optical image of the (specific) surface area being sharper and/or comprising more optical information about the surface are than the OCT 3D image or the 3D optical image individually. The surface area of the OCT image and the surface area captured with the imaging unit may partially, widely or completely overlap. In the area where the OCT 3D image and the 3D optical image overlap the processed 3D image (based on the OCT 3D image and on the 3D optical image) may be sharper and/or provide more optical information than the OCT 3D image or the optical 3D image do individually. According to an exemplary embodiment of the endoscope system the endoscope tube has a two-lens objective providing a separate use of the first lens, and the second lens, wherein the first lens supplies the imaging unit and wherein the second lens supplies the OCT unit.

It may be appropriate in terms of cost efficiency to use a two-lens objective where the OCT unit has its own lens, the first lens of the two-lens objective, for the OCT beam. The imaging unit may have another, the second lens, for capturing images.

According to an exemplary embodiment of the endoscope system, wherein the second lens supplying the OCT unit is further adapted to illuminate the sample with visible light.

The use of a light source being with a semitransparent mirror feeding the visible light to the OCT sample arm may provide an illumination source at the end of the endoscope tube to illuminate the sample with the second lens, or OCT lens being also adapted to direct visible light.

According to an exemplary embodiment of the endoscope system, the OCT image being a 3D representation of a surface area, and the image being generated with the imaging unit, are processed to represent a 3D image of the surface area.

The further processing of the OCT image and the image being generated with the imaging unit may apply to using one lens as an OCT lens and the other lens as an imaging lens (for imaging with visible light), so that the 3D OCT image and the 2D image from the imaging unit are combined and processed to generate a 3D image from the surface of the sample.

When the optics systems for the OCT imaging and the camera imaging are separated there may be an individual color-correction for their respective spectral bands. Both optics systems may have a fixed relation in their respective focal distances, e.g. to have the best (or optimized) camera focus on the tissue surface and the best focus for OCT in a certain depth in the tissue (e.g. 1 mm below the tissue surface)

Any of the described optics systems may be equipped with an electronically controlled focusing element, e.g. a liquid lens, allowing changing a focal plane/a working distance for both modalities (the OCT unit and the vision unit) at the same time.

According to an exemplary embodiment, the endoscope system further comprises a screen displaying one or more images based on data from the OCT unit and/or data from the imaging unit.

In general, an image based on data from the OCT unit may be a B-scan, an en-face image or a 3D rendered image.

Furthermore, on the screen may be displayed one or more of the group of: the OCT image in a 3D rendering, the OCT image and the en-face image combined, the OCT image as false color overlay, a scan line location indicated in the en-face image, a 2D OCT FOV (Field of View) indicated in the en-face image.

A screen coupling to the imaging unit and to the OCT unit may display the results for the operating doctor. The results may be arranged in a regular way side by side of each other or one above the other. As a further alternative the results of both units may be arranged one behind another so that the operating doctor may understand the importance of the result by looking only to one screen.

According to an exemplary embodiment of the endoscope system the endoscope tube is coupled to a handle, or alternatively to a robotic arm for moving the endoscope tube.

Support for handling or moving the endoscope tube may be supported by a handle which an operating doctor may use or by a robotic arm. Using the handle or the robotic arm may facilitate to bring the objective at the distal end of the endoscope in the right position. Further, the handle and the robotic arm may allow for rotating the endoscope tube by 90° so that requirements for the OCT unit may be fulfilled. Thus, moving of the endoscope tube may comprise displacing and/or rotating. According to a translational motion of the endoscope tube or tube head the field of view may enlarge if the endoscope tube or tube head approximates the sample, and the field of view may diminish in size if the endoscope tube or tube head may diminish if the distance from the sample increases. According to a rotary motion a horizon may raise.

According to an exemplary embodiment the endoscope system, the OCT unit comprises at least one device of a first group of NIR, VIS, SLED light source (super-luminescent diode), swept source laser, FDML laser (frequency-domain mode-locked laser), super-continuum light source (VIS) for the light source,

of a second group of 1D or 2D scanning, resonant scanning, closed-loop, combination of resonant and closed loop, rotating prism scanner for a scanning element, and/or
of a third group of a spectrally resolved spectrometer with line sensor or a time resolved spectral acquisition using a swept source and a photo detector like e.g. an avalanche photo diode APD, or a silicon photo-multiplier SiPM to record an A-scan.

According to an exemplary embodiment of the endoscope system,

the imaging unit comprises at least one device of the group of 2D/3D, extended depth of field imaging (EDOF), light field imaging, pupil plane encoding, camera CCD, camera CMOS, laser scanning, VIS, NIR, fluorescence imaging, hyperspectral imaging.

Any of the images displayed on the screen may be generated by stitching.

The term “processed date” or “processing data” by the OCT unit is not limited to the ways of processing already being described. Moreover, the term “processing” of the OCT unit may include: localization of interfering contours by using short-range-LIDAR, distance measurements, determination of tissue properties (e.g. elastography, density), recognition and registration of tissue structures for the purpose of navigation, detecting of structures under the surface of the tissue (e.g. blood vessels, nerves), determination of healthy tissue and tumor tissue.

Applications may be in but not limited to visceral surgery, gastro-intestinal surgery, brain surgery, laparoscopy, or colonoscopy.

### BRIEF DESCRIPTION OF THE DRAWINGS

FIG. 1 shows schematically an assembly for optical coherence tomography (OCT) according to the state of the art.

FIG. 2 shows schematically an endoscope according to the state of the art.

FIG. 3 shows a perspective/schematic view of an embodiment of an endoscope system.

FIG. 4 is a schematic drawing of the end section of a flexible tube for 2D having one end lens.

FIG. 5 is a schematic drawing of an end section of a flexible endoscope tube for 2D having two end lenses.

FIG. 6 is a schematic drawing of an end section of a flexible endoscope tube for 3D having two end lenses.

FIG. 7 is a schematic drawing of an end section of a flexible and rigid endoscope tube for 3D having three lenses.

FIG. 8A is a schematic drawing of a complete rigid endoscope tube for 3D having three end lenses.

FIG. 8B is a schematic drawing showing a lens arrangement with small diameter.

FIG. 8 C shows a schematic drawing of an offset compensation.

FIG. 8 D shows an endoscope tube with a mutual optical path for illumination and the OCT sample arm.

FIG. 9 shows an overview of different embodiments of an endoscope system.

FIG. 10 shows a schematic and perspective view of the endoscope head and of a surgical tool.

FIG. 11 A shows a schematic view of a bending endoscope head.

FIG. 11 B shows a schematic view of an endoscope head extending straight.

FIG. 12 A shows a schematic view of a turned endoscope head.

FIG. 12 B shows a drawing representing changing spots when an endoscope head is turning.

FIG. 13 A shows a schematic view of a sample and the direction of an A-scan.

FIG. 13 B shows a direction of a B-scan extending along the x-axis and a result.

FIG. 13 C shows a drawing representing changing spots when an endoscope head is turning.

FIG. 13 D shows a drawing representing scan on an area spanned by the x- and y-axis.

FIG. 13 E shows a schematic 3D view of one C-scan.

FIG. 13 F shows a schematic 3D view of three C-scan.

### DETAILED DESCRIPTION

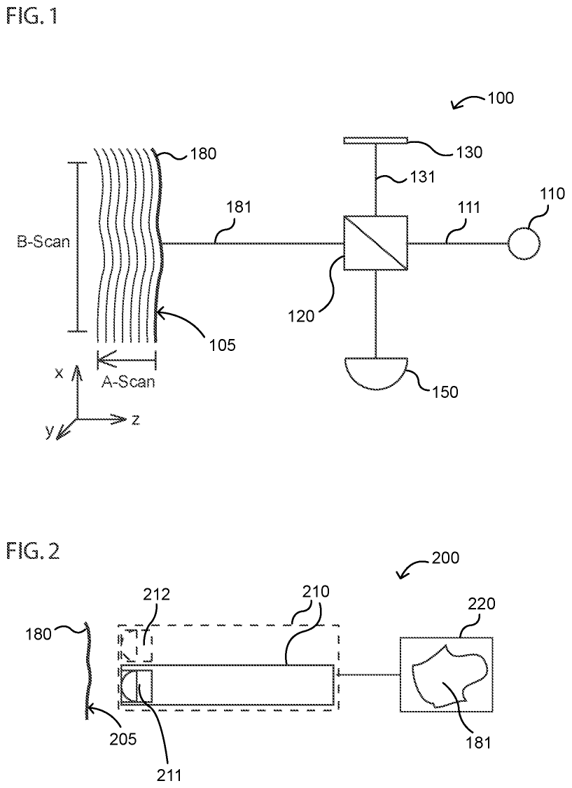

FIG. 1 shows schematically an optical coherence tomography setup 100 according to a state of the art. A light source 110 produces a light beam 111 which falls into a beam splitter 120. The beam splitter 120 partially let pass through the light beam 111 towards a sample 180 and partially reflects the light beam 111 towards a reference mirror 130. A first reflected light beam 131 coming from the reference mirror 130 passes partially directly through the beam splitter 120 onto a detector 150. A second reflected light beam 181 coming from the sample 180 is at least partially reflected from the beam splitter 120 and thus also propagates towards the detector 150. Both, the first light beam 131 and the second light beam 181 interfere in the detector 150. A deep scan into the sample, or so called “A-Scan”, showing an area of the sample 180 in depth, may be based on a calculation in a frequency domain (abbr. “FD-OCT”) or in a time domain (abbr. “TD-OCT”), and may be displayed on a screen.

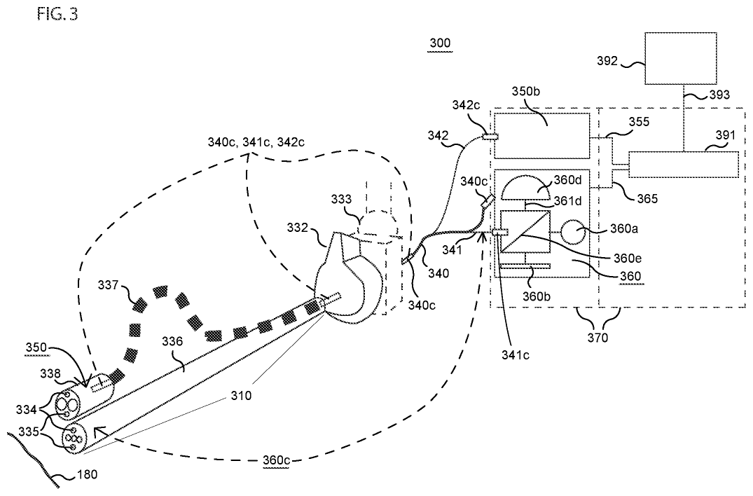

FIG. 2 shows an endoscope 200 according to a state of the art comprising an endoscope tube 210 providing a two-dimensional (2D) image 181 of the surface of a sample 180 based on a one-lens optic 211. By providing a further lens 212 a three-dimensional (3D) image 181 may be recorded and shown on a display 220.

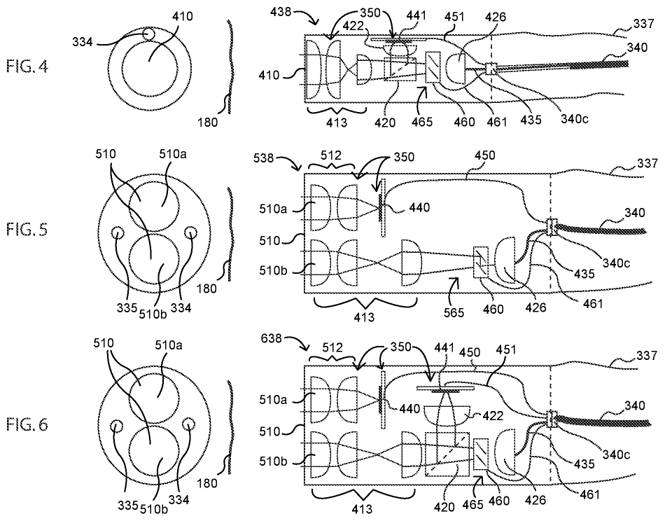

FIG. 3 shows an embodiment of an endoscope system 300 comprising an endoscope tube 310 (in a perspective view), and an imaging unit 350 (see also FIG. 4 to FIG. 8), and an OCT unit 360 (in a schematic view).

The OCT unit 360 has a light source 360a, a beam splitter 360e, a reference mirror 360b and a detector 360d. A sample arm 360c of the OCT unit 360 extends to and is at least partially arranged within the endoscope tube 310.

The beam splitter 360e partially let pass through a light beam generated by the light source 360a towards a sample 180 and partially reflects the light beam towards the reference mirror 360b. The light beam being reflected towards the sample 180 is guided by the sample arm 360c being at least partially arranged inside the endoscope tube 310. A first reflected light beam comes back from the reference mirror 360b and passes through the beam splitter 360e to the detector 360d. A second reflected light beam comes back from the sample 180 and (passing the sample arm 360c) is partially reflected from the beam splitter 360e to the detector 360d. Both reflected light beams (coming from the sample 180, and from the reference mirror 360b, respectively) interfere on the detector 360d. Based on the detected interference signal a B-Scan may be generated if the sample 180 is scanned along a line. If the sample 180 is scanned over an area, a C-Scan may be generated based on the detected interference signal. An OCT unit cable 341 being an integral part of the sample arm 360c may couple towards the endoscope tube 310. The OCT unit cable 341 may be exchangeable by means of connectors 340c, 341c.

The imaging unit 350 being at least partially located within the endoscope tube 310 may particularly be arranged within the rigid tube head 338, being a distal end part of the endoscope tube 310. The imaging unit 350 may at least comprise a lens or lens arrangement and an image acquisition device (see FIG. 4 to FIG. 8), e.g. a CCD chip. The imaging unit 350 may further comprise an image processing unit 350b receiving captured imaging data. An imaging unit cable 342 may couple to the endoscope tube 310 and to the image processing unit 350b via a connector 342c for submitting imaging data. However, as an alternative, acquired image data may also be wirelessly submitted and/or preprocessed already inside the tube head 338, or inside the endoscope tube 310, so that an imaging unit cable 342 may be omitted in case of a wireless submission of the imaging data.

The OCT unit 360 may be a stand-alone device or may be arranged in a central unit 370. Although being at least partially arranged within the endoscope tube 310, the imaging unit 350 may couple or extend to an image processing unit 350b for processing imaging data. Both, the OCT unit 360 and the image processing unit 350b may be stand-alone devices or may both be arranged in a central unit 370, which may additionally comprise a computer 391. The computer 391 may couple to the OCT unit 360 with an OCT cable 365, and may couple to the image processing unit 350b with an imaging cable 355. The computer 391 may transmit data to a screen 392 with a screen cable 393.

A connection cable 340 coupling the endoscope tube 310 and the central unit 370 may provide combined data and beam guides for the imaging unit 350 and the OCT unit 360. The connection cable 340 may include the OCT unit cable 341 and the imaging unit cable 342. Alternatively, the connection cable 340 may branch to the OCT unit cable 341 and to an imaging unit cable 342 at any point. Connectors 340c, 341c, 342c may provide a detachable connection for any cable required for the OCT unit 360 and the imaging unit 350.

The light beam emitted by the light source 360a going to the sample 180, and the reflected light beam coming from the sample 180 and going to the beam splitter 360, hence, both pass the endoscope tube 310. Because of this, the connection cable 340 may comprise a fiber optic cable (also see FIG. 4-8, 435) which couples to the OCT unit cable 341 which comprises a fiber optic cable, as well. Hence, also the connection cable 340 is an integral part of the sample arm 360c of the OCT unit 360.

The endoscope tube 310 may be a rigid endoscope tube 336, or as an alternative built as a flexible endoscope tube 337. The endoscope tube 310 has a tube head 338 at its distal end, the tube head 338 being rigid and pointing towards the sample 180. The light beam may leave the endoscope tube 310 from the tube head 338 towards the sample 180. Furthermore, the tube head 338 may be adapted to scan the sample 180 along a line (for generating a B-Scan) or adapted to scan the sample 180 over an area (for generating a C-Scan). At least one light source 334, 335 at the distal end of the endoscope tube 310, or the tube head 338, respectively, may provide illumination for the sample 180.

As an alternative, light for illumination of the sample 180 may be generated by a light generator (not shown) from outside, or at the proximal end of the endoscope system 300 and the light may be directed to the handle 332 by a light guide.

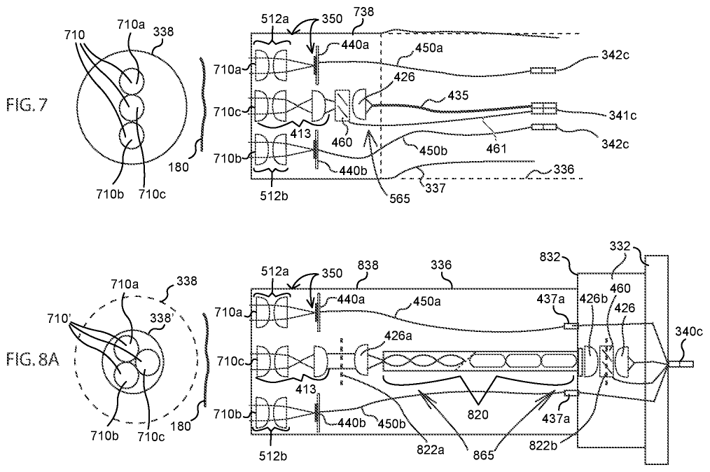

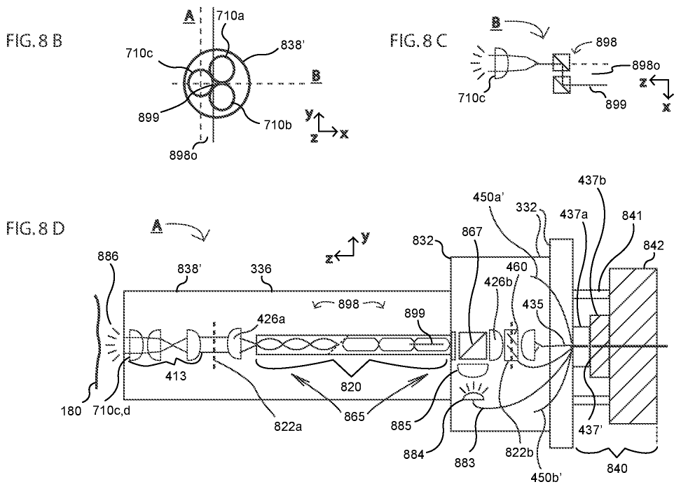

From the handle 332 the light for illumination of the sample 180 may be guided inside the endoscope tube 300 by a separate light guide towards the distal end of the endoscope tube 310, or tube head 338, where constituting the light source 334, 335. As an alternative, the light for illumination of the sample 180 may be guided within the OCT sample arm towards the distal end of the endoscope tube 310, or tube head 338 (described in more detail with FIG. 8 D).

Handling of the endoscope tube 310 may be accomplished by a handle 332 adapted to being moved by an operating doctor, or alternatively by a robotic arm 333 for an automated movement control of the endoscope tube 310.

FIG. 4 is a schematic cross-sectional drawing of an endoscope head 438 of a flexible endoscope tube 337 providing 2D visual imaging and an OCT generated B-scan and C-scan based on one capturing lens 410 as an outmost part of an objective 413 (see also 410 left part of FIG. 4). A light beam coming from the OCT unit 360 (or “OCT beam”) is supplied by a fiber optic cable 435 into the endoscope head 438. The OCT beam passes an OCT lens 426 and is deflected by a micro scanner 460 before passing through a combined beam splitter 420. After traversing the combined beam splitter 420 the OCT beam passes the objective 413 in the direction towards the sample 180 where the OCT beam is reflected. The micro scanner 460 allows for scanning specified regions of the sample 180, so that, particularly, the reflected OCT beam may contain information about different regions of the sample 180 to which the OCT beam (in the direction to the sample 180) was previously directed by the micro scanner 460. Scanner control signals are provided to the micro scanner 460 by a scanner control cable 461 which connects to a connector 340c receiving the control signals provided from the OCT unit 360. After being reflected from the sample 180 and going through the objective 413 again, the beam splitter splits this reflected beam into a portion going to the OCT unit 360, and to a portion going to the imaging unit 350. Hence, the OCT beam coming from (and going to) the OCT unit 360 runs through the combined beam splitter 420. The just described OCT part of the embodiment in FIG. 4 is identical with the OCT parts of the embodiments shown in the figures FIG. 5 to FIG. 7. Therefore, the reference signs are chosen identical and repetitions of these same constructive and functional parts are omitted for the FIG. 5 to FIG. 7. In particular, the imaging unit 350 may comprise a lens 410, 413 and at least one or two image acquisition devices, such as CCD chips 440, 441, 440a, 440b, being depicted with FIG. 4 to FIG. 8.

As (for FIG. 4) indicated above, the combined beam splitter 420 partially deflects or reflects light coming from the sample 180 towards the imaging unit 350. At first, the beam may be directed to a chip lens 422 and then to a photo chip 441. The photo chip 441 generates an electrical vision signal which is conducted to the connector 340c via a photo chip line 451. In sum, the connector 340c may couple the input OCT beam (generated by the OCT unit 350, see FIG. 3) and the output OCT beam (reflected by the sample 180, see FIG. 3), the OCT control signal (controlling the micro scanner 460), and the vision signal towards the connection cable 340, which said signals each runs in an individual and specified cable.

As can be seen in FIG. 4, light coming from the sample 180 falls on the beam splitter 420, which divides the light beam into the OCT beam component and the vision beam component. As a consequence, the OCT unit 360 and the imaging unit 350 share the same objective 413 which may consist only of a capturing lens 410 for capturing the reflected light. Hence, the endoscope head 438 has the capturing lens 410 in combination with the beam splitter 420 for providing a shared use of both, the capturing lens 410 and the objective 413.

As already mentioned, the specific OCT parts described with FIG. 4 are also shown and used for the embodiments shown in FIG. 5 to FIG. 7. These same components are, therefore, marked with identical reference signs, and a detailed description repeating the same subject matter is omitted.

An endoscope head 538 shown in FIG. 5 has a first lens 510b representing the single capturing lens 410 and being a part of a first objective 413 as depicted in FIG. 4 providing a channel for the OCT beam. However, the endoscope head 538 shown in FIG. 5 differs from the endoscope head 438 shown in FIG. 4 slightly: There is a second capturing lens 510b as a part of a second objective 512 and may be used to provide a 2D visual image again. A two-lens objective 510 comprising the first capturing lens 510a and second capturing lenses 510b thus provides two separated channels, one for the imaging channel and one for the OCT channel. There is no beam splitter 420 needed in the endoscope head 538 as described with FIG. 4. A photo chip 440 may capture the vision signal captured by the second capturing lens 510b. A photo chip line 450 leads the electronic vision signal generated by photo chip 440 towards the connector 437, which again couples to the connection cable 339. By omitting combined beam splitter 420, the space of the endoscope head 538 is less densely packed but the tube head 538 may have a greater diameter compared to the endoscope head 438, described in FIG. 4.

FIG. 6 is a schematic drawing of an endoscope head 638 which combines the structure and functions given by the endoscope heads 438 and 538 being described with FIG. 4 and FIG. 5, respectively. The endoscope head 638 may be used for generation of a 3D image. Firstly, the elements from endoscope head 438 shown in FIG. 4 are completely integrated for supplying a combined 2D vision and OCT signal output, by using a first capturing lens 510a (and a first objective 413) as a part of the two-lens objective 510. As a consequence, this part may already provide a shared use of the first capturing lens 510a, and the shared objective 413. Further, a second capturing lens 510b (and second objective 512, like in FIG. 5) may provide a further image input for the imaging unit 350. Hence, the first lens 510b in shared use and the second lens 510a may provide data for the imaging unit 350 which may generate a 3D image to be displayed on the screen 392.

An endoscope head 738 shown in FIG. 7, again uses an OCT lens 710c as a part of the objective 413 for providing an OCT beam like in any other of the preceding embodiments as depicted in FIG. 4 to FIG. 5. However, the endoscope head 738 shown in FIG. 7 differs slightly from the endoscope head 538 as shown in FIG. 5 by using two separate lenses 710a, 710b for completing an input for the imaging unit 350 for generating a 3D image. The 3D image being captured by the two separate lenses 710a, 710b (and two separate objectives 512a, 512b) has an independent channel compared to the OCT channel coming the OCT lens 710c for generating an OCT scan (in the form of a B-Scan). Consequently, the objective being used is a three-lens objective 710 for generating the 3D image by the imaging unit 350 and the B-Scan by the OCT unit 360. A first vision line 452a transfers data coming from the first separate lens 710a (as a part of a first separate objective 512a) towards a connector 739. A second vision line 452a transfers data coming from the second separate lens 710b (as a part of a first separate objective 512b) towards a connector 739, respectively. Connectors 437a may couple separately or be grouped together to the connector 437a.

In FIG. 7 a dashed line marks that it is possible to use a rigid tube 336 instead of having a flexible tube 337. Generally, in any of the embodiments described with FIG. 4 to FIG. 7 the flexible tube 337 is replaceable by a rigid tube 336, since data and fiber cable being used work as well in a rigid tube 336 as in a flexible tube 337.

FIG. 8A shows an embodiment which is based on the identical three-lens objective 710 described in connection with FIG. 7. The embodiment shown in FIG. 8A, however, differs therein that a position of the micro scanner 460 may be shifted outside of an endoscope head 838, or away from the three-lens objective 710, respectively. This is achieved by a rigid insert forming a relay optical system consisting of a so-called GRIN and/or tube lens objective 820 in combination with a first lens 426a and a second lens 426b joining on alternating ends of the GRIN and/or rod lens objective 820. The GRIN and/or rod lens objective 820 may provide a first pupil plane 822a and a second pupil plan 822b being identical and being located on alternating sides of the GRIN and/or rod lens objective 820. The second pupil plane 822b may extend through the mid of the micro scanner 460. In the direction towards the OCT unit 360, the OCT beam may cross the OCT lens 426. The micro scanner 460 may already be located within an intermediate piece 832 between the endoscope tube 838 and the handle 332 (or robotic arm 333). As an alternative, the micro scanner 460 may be already located within the handle 332 (or robotic arm 333) without the use of an intermediate piece 832.

Summarizing, there is an OCT optic 465 shown in FIG. 4 and FIG. 6 using a beam splitter 420. The corresponding tube heads 438, 638 may provide a higher integration and a smaller diameter. Whereas an OCT optic 565 shown in FIG. 5 and FIG. 7 using no beam splitter 420 may have less integration and the respective tube heads 538, 738 may have a greater diameter. An OCT optic 865 shown in FIG. 8A is similar to the OCT optic 565 of FIG. 7 with an exception, that GRIN/rod lens systems (820) are used for allowing that the micro scanner 460 is placed away from the tube head 838 towards an end of the endoscope tube 336 or even in an intermediate piece 832 or into the handle 332, or into the robotic arm 333.

Depending on the use of different objectives, the endoscope tube 310 may have a diameter in the range between 5 mm and 12 mm, or even less. In particular, the lenses 710a, 710b, 710c may be arranged in greatest possible proximity to each other, so that the diameter of the endoscope head 338′ may be reduced by this type of lens 710′ (FIG. 8A) instead of arranging the lenses 710a, 710b, 710c in a row like shown in lens 710 with FIG. 7, where the diameter of the endoscope head 338 is greater.

FIG. 8B shows again the three optical lenses 710a, 710b, and 710c being arranged in greatest possible proximity to each other, where the diameter of the endoscope head 838′ is small. Dashed straight line A indicates a cross-section through the lens 710c which is a part of the (OCT) sample arm (see 360c FIG. 3) which is shown in FIG. 8D in more detail. The lens 710c is arranged in the middle of the endoscope tube regarding the cross-section A which extends along a y-axis. Further, a dashed line B indicates a cross-section extending in the direction of an x-axis. The lens 710c is shifted with an offset 898o with respect to a middle axis 899 of the endoscope head 838′.

FIG. 8C shows schematically the cross-sectional view as indicated by the dashed line B (in FIG. 8B), so that the offset 898o of the lens 710c with respect to the middle axis 899 of the endoscope head 838′, or of the endoscope tube 336 (both shown in FIG. 8D) is apparent. A shift compensation 898 in the direction of the x-axis may have any form or being achieved by any optical method so that the optical offset 898o of the lens 710c is compensated. The optical path of the lens 710c is shifted or compensated so that the optical path is then identical with the middle axis 899 of the endoscope head 838′, or of the endoscope tube 336, respectively.

FIG. 8D is a schematic drawing as indicated by dashed line A in FIG. 8B. The drawing shows a rigid endoscope tube with a mutual optical path for illumination and the (OCT) sample arm (360c, see FIG. 3), wherein the optical paths for generating the regular mono or stereo images are omitted, so that only the optical path of the OCT sample arm (again 360c) is shown. Therefore, FIG. 8D only shows an OCT lens 710d of the OCT sample arm and not one or two other lenses 710a, 710b of the stereo imaging or regular 2D imaging path. Within the optical path of the OCT sample arm a semitransparent mirror 867 allows for transmitting the OCT information and additionally allows for coupling in light for an illumination 886 of the specimen 180 into the optical path of the OCT sample arm. At the distal end of the OCT sample arm (360c) the OCT lens (710d) may be adapted to illuminate the sample 180. With this function, the OCT lens (710d) represents also an illumination source (710d) for illuminating the sample 180 with visible light. The light is generated by a light source 884 providing a sufficient intensity and the light source 884 may be controlled and powered via a twistable connector 437′. The light source 884 may be a halogen light source, a Xenon light source, an LED light source, a laser light source, or the like. As an alternative, the light source may be a light guide being fed by a light generator (not shown) being arranged outside of the handle. In addition, to controlling and powering the light source 884, the twistable connector 437′ may also control and power the photo chip lines 450 a, b for generating a stereo image. The twistable connector 437′ may comprise a first, stationary part 437b being coupled to a stationary holder 842, and a second rotatable part 437a being coupled to the handle 332. Further, the stationary holder 842 comprises a drive 841 so that the handle (with the rotatable part 437a) is rotatable in relation to the stationary part 437b, or the holder 842, respectively. Since the light generating light source 884 being located in the handle 332 and the OCT sample arm 360c and the light path for illumination share the same lenses or the same lens path, the diameter of the endoscope head 838′ may be even more reduced compared to having light sources near the distal end of the endoscope head 838.

Further, since the cross-sectional view along dashed line A (in y-direction) does not show the offset 898o, the offset compensation 899 is only indicated as being located within the optical path of the OCT sample arm somewhere between the twistable connector 437′ and the distal end of the endoscope tube 336. In particular, the offset compensation 899 may be located between the fiber optical cable 435 connecting to the twistable connector 437′ and the distal end of the endoscope tube 336, or of the endoscope head 383′, respectively. An axis of the twistable connector 437′ may be identical with the middle axis 899 of the endoscope tube 336, or of the endoscope head 838′, respectively.

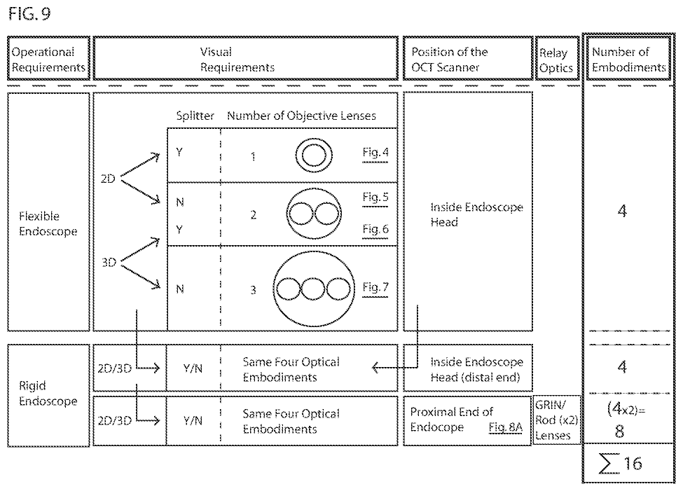

FIG. 9 shows an overview of different embodiments of an endoscope system as being described from FIG. 4 to FIG. 8D. Various other embodiments are possible. Depending on requirements of an operation it may be appropriate to use a flexible endoscope tube or a rigid endoscope tube. Further, depending on visual requirements 2D or 3D imaging may be demanded. If a larger diameter of the endoscope tube is critical for the operation it may be appropriate to use a beam splitter. Otherwise, if the diameter of the endoscope tube is not critical no beam splitter may be used for still getting a 3D image. FIG. 4 describes a one-lens optic having the smallest diameter. FIG. 5 and FIG. 6 both depict a two-lens optic having a greater diameter. FIG. 7 describes a three-lens optic requiring an even greater diameter than the two-lens optics from FIG. 5 and FIG. 6. Moreover, it may be appropriate to have one or more joints where, e.g. the complete endoscope tube may be exchangeable. As an alternative, the position of the OCT scanner may be located outside and behind, or at the end of the endoscope tube. This, however, may require that the endoscope tube is of a rigid type. When using a rigid endoscope tube, a GRIN and/or rod lens optic (×2) may be used to partially transfer the OCT beam through the endoscope tube. In sum, at least 16 different variations depending on so defined requirements may be derived from the 5 embodiments from FIG. 4 to FIG. 8D being summarized in FIG. 9.

By referring to FIG. 4 and FIG. 6 the table in FIG. 9 illustrates a use of the beam splitter with “Y”, so that the OCT beam and the optical information for the imaging unit are transmitted mutually on the same path within the endoscope tube.

In contrast, and now referring to FIGS. 5, 7 and 8A, the semitransparent mirror (see 867 in FIG. 8D) may be used to use the OCT lens also for illumination of the sample, as being described with FIG. 8D. This may lead to even more variations of the endoscope system.

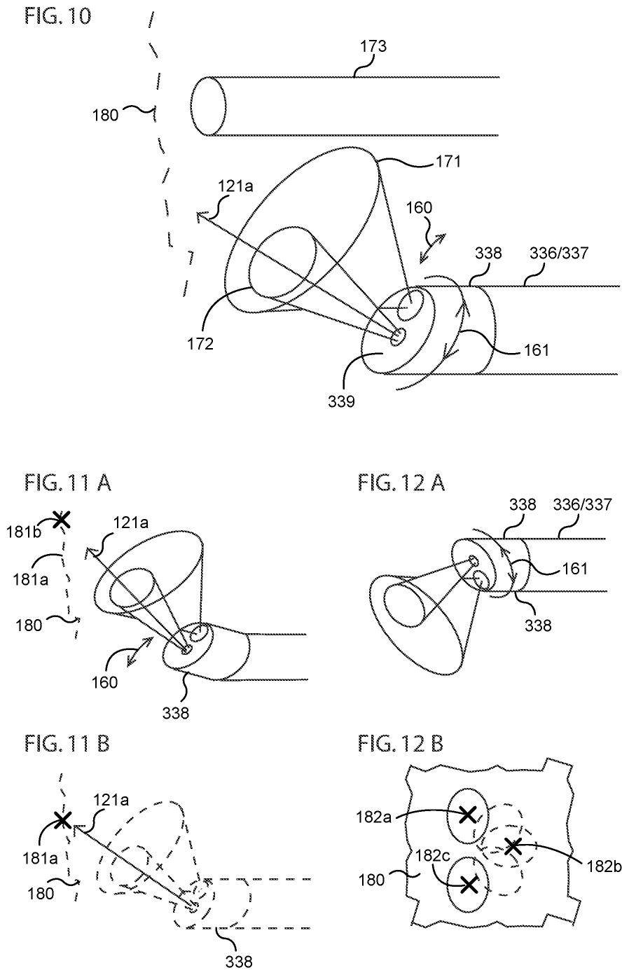

FIG. 10 shows a schematic and perspective view of an endoscope head 338 and of a surgical tool 173 directed a sample 180. From a sloped distal end surface 339 of the endoscope head 338 a visual field 171 of imaging unit 350 (see FIG. 3) and an OCT visual field 172 may have the same (central) focus direction 121b. However, a visual field 171 of imaging unit 350 may be larger (or broader) than an OCT visual field 172. The endoscope head 338 may mutually turn with a rotatable endoscope tube 336 being represented with arrow 161. A further arrow 160 depicts that the endoscope head 338 may bend relative to the endoscope tube 336.

FIG. 11A shows a schematic view when an endoscope head 338 bends relative to the endoscope tube 336, 337. The focus direction 121a initially pointing to a first center point 181a on the sample 180 may change so that the focus direction 121a then points towards a second center point 181b on the sample 180. FIG. 11 B just shows as a reference the focus direction 121 pointing towards the first center point 181a when the endoscope head 338 is not angled.

FIG. 12A shows a schematic view of an endoscope head 338 turning mutually with the endoscope tube 336 depicted by arrow 161. FIG. 12B shows a drawing representing the changing spots when the endoscope head 338 is rotated. The focus direction 121a (see FIG. 10) may change from a first center point 182a on the sample 180 continuously via a second center point 182b, and continuously towards a third center point 182c. As a consequence, the area which is covered by the OCT visual field 172 (see FIG. 10) may be extended.

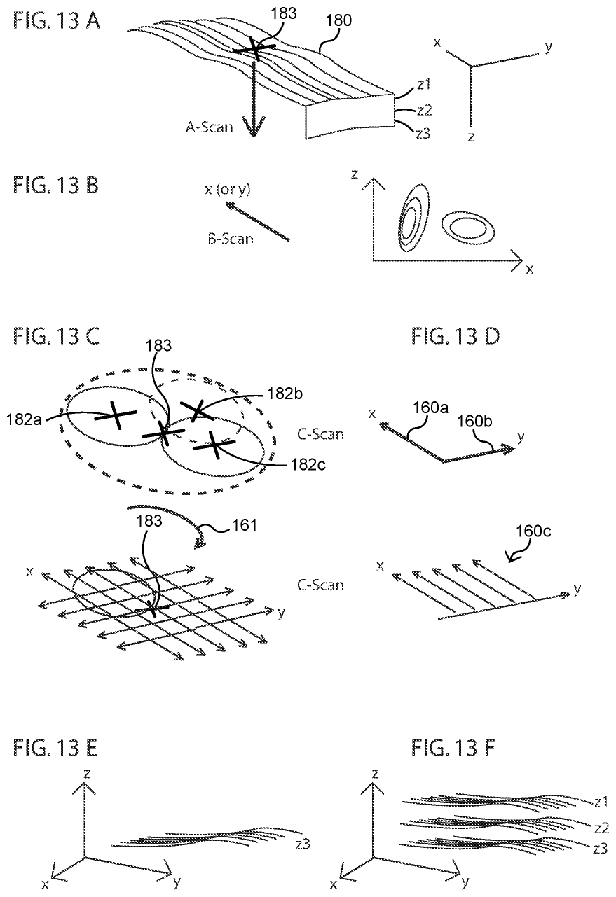

FIG. 13A shows a schematic view of the sample 180 and the direction of an A-scan going basically perpendicular to a main area of the sample 180. The direction of the A-scan is usually defined as the z-axis, whereas the main area of the sample may be defined by the area spanned by the x- and y-axis. FIG. 13B illustrates that a B-scan is made when the A-scan moves along a line at least partially extending along the x-axis or y-axis. Since information about properties of the sample 180 are already provided by the A-scan the information gathered comes from a cross-section. Values derived from this cross-sectional information (in direction z- and x- and/or y-axis) may be 2-dimensional displayable using contour lines.

FIG. 13C shows a perspective drawing showing changing spots when an endoscope head (as being described with FIG. 10) is rotating according to an arrow 161. FIG. 13C is merely a perspective view of the changing direction of the focus direction 121a (as being described with FIG. 10, FIG. 12A, and FIG. 12B. An area spanned by the x- and y-axis may be partially covered by the OCT visual field 172 having a moving center point 182a, 182b, 182c.

FIG. 13D shows a drawing representing a scan on an area spanned by the x- and y-axis. This may be another way of directing the A-scan over the sample 180 starting in direction along a line of the x-axis and then continuously shifting these lines going along the x-axis 160a traversal along the y-axis 160b. This spans a very regular area 160 in a form of a C-scan extending perpendicular to the A-scan (or z-axis). FIG. 13 E shows a schematic 3D view of one C-scan. Since information for the C-scan is already gathered from a volume at least partially extending in all directions (x-y-z) is difficult to represent. However, there may be options showing a perspective view of one C-scan (see also FIG. 13 A. Further, a certain number (here “3”) of C-scans (z1, z2, z3) may be represented on a screen in a similar way by spacing e.g. the three perspective views (z1, z2, z3) of the C-scan (see FIG. 13 F). It may also be appropriate to display a holographic image.

1. An endoscope system (300) for imaging a sample (180), an inner part of a patient, or an organ, wherein the endoscope system (300) comprises:
an endoscope tube (310),
an imaging unit (350) for imaging the inner part of the patient, wherein the imaging unit (350) is at least partially located inside the endoscope tube (310), and
an optical coherence tomography unit (360; OCT unit), wherein said imaging unit (350) is distinct from the OCT unit (360), and
wherein a sample arm (360c) of the OCT unit (360) is at least partially located inside the endoscope tube (310).

2. The endoscope system (300) according to claim 1, further comprising a screen (392) for displaying one or more images based on processed data from of the OCT unit (360) and/or data from the imaging unit (350).

3. The endoscope system (300) according to claim 1, further comprising an OCT unit cable (341), and/or an imaging unit cable (342), wherein
the OCT unit cable (341) comprises a fiber optic cable as a part of the sample arm (360c) of the OCT unit (360), and wherein
the imaging unit cable (342) couples the endoscope tube (310) with an image processing unit (350b).

4. The endoscope system (300) according to claim 1, comprising a connection cable (340), wherein the connection cable (340) comprises the OCT unit cable (341) and the imaging unit cable (342).

5. The endoscope system (300) according to claim 1, further comprising at least one connector (340c; 341c; 342c) mounted at an end of at least one of the group of the endoscope tube (310), the connection cable (340), the OCT unit cable (341), and the imaging unit cable (342), so that the endoscope tube (310) is separable from at least one of the group of the OCT unit (360) and the image processing unit (350b).

6. The endoscope system (300) according to claim 1, wherein the endoscope tube (310) comprises a rigid section (336), and a micro scanner (460) being located in said rigid section (336), and/or mounted to said rigid section (336), wherein the micro scanner (460) is adapted to scan the sample (180) in one and/or two dimensions.

7. The endoscope system (300) according to claim 1, wherein the endoscope tube (310) comprises a flexible section (337), wherein the flexible section (337) is located behind a rigid tube head (338) seen from a distal end of the endoscope tube (310), and wherein the micro scanner (460) is located in the rigid tube head (338) or attached to the rigid tube head (338).

8. The endoscope system (300) according to claim 1, wherein the endoscope tube (310) comprises an illumination source (334, 335, 710d) at its distal end and wherein the illumination source (334, 335, 710d) is adapted to illuminate the sample (180) with visible light.

9. The endoscope system (300) according to claim 1, wherein the endoscope tube (310) comprises a capturing lens (410) and a beam splitter (420), providing a shared use of an objective (413) for the OCT unit (360) and the imaging unit (350), so that an OCT image can be generated with the OCT unit (360), and a 2D image can be generated with the imaging unit (350).

10. The endoscope system (300) according to claim 1, wherein the endoscope tube (310) comprises a first capturing lens (510a), and a second capturing lens (510b), and a beam splitter (420), providing a shared use for one the one of the first and/or second capturing lenses (510a, 510b), for the OCT unit (360) and/or the imaging unit (350), so that an OCT image can be generated with the OCT unit (369), and a 3D image can be generated with the imaging unit (350).

11. The endoscope system (300) according to claim 1, wherein the endoscope tube (310) has two parallel objective lenses (510a, 510b) providing a separate use of the first objective lens (510a), and the objective second lens (510b), wherein the first lens (510a) supplies the imaging unit (350) and wherein the second lens (510b) supplies the OCT unit (360).

12. The endoscope system (300) according to claim 11, wherein the second lens (510b, 710d) supplying the OCT unit (360) is further adapted to illuminate the sample (180) with visible light.

13. The endoscope system (300) according to claims 9 to 11, wherein the OCT image being a 3D representation of a surface area and the image being generated with the imaging unit (350) are processed to represent a 3D image of the surface area.

14. The endoscope system (300) according to claim 1 or 2, further comprising a screen (392) displaying one or more images based on data from of the OCT unit (360) and/or data from the imaging unit (350).

15. The endoscope system (300) according to claim 1, wherein the endoscope tube (310) is coupled to a handle (332), or alternatively to a robotic arm (333) for moving the endoscope tube (310).

16. The endoscope system (300) according to claim 1, wherein the OCT unit (360) comprises at least one device of a first group of NIR, VIS, SLED light source (super-luminescent diode), swept source laser, FDML laser (frequency-domain mode-locked laser), super-continuum light source (VIS) for the light source, of a second group of 1D or 2D scanning, resonant scanning, closed-loop, combination of resonant and closed loop, rotating prism scanner for a scanning element, and/or of a third group of a spectrally resolved spectrometer with line sensor, a time resolved spectral detection with a photo detector for swept source, for an A-scan detector.

17. The endoscope system (300) according to claim 1, wherein the imaging unit (350) comprises at least one device of a group of 2D/3D, extended depth of field imaging (EDOF), light field imaging, pupil plane encoding, camera CCD, camera CMOS, laser scanning, VIS, NIR, fluorescence imaging, and/or hyperspectral imaging.

18. A method of operating an endoscope system (300) for imaging a sample (180), an inner part of a patient, or an organ, comprises:
providing an endoscope tube (310),
providing an imaging unit (350) for imaging the inner part of the patient, wherein the imaging unit (350) is at least partially located inside the endoscope tube (310), and
providing an optical coherence tomography unit (360; OCT unit), wherein said imaging unit (350) is distinct from the OCT unit (360), wherein a sample arm (360c) of the OCT unit (360) is at least partially located inside the endoscope tube (310), and
processing data from the imaging unit (350) and/or from the OCT unit (360), so that the processed data are displayable on a screen (392).
